(19) United States
(12) Patent Application Publication
(10) Pub. No.: US 2023/0124816 A1
(43) Pub. Date: Apr. 20, 2023

(54) SURGICAL SUTURE HAVING EXCELLENT BIOCOMPATIBILITY AND LOW FRICTION, AND METHOD FOR MANUFACTURING SAME

(71) Applicant: SOGANG UNIVERSITY RESEARCH & BUSINESS DEVELOPMENT FOUNDATION, Seoul (KR)

(72) Inventors: Kwan Woo SHIN, Seoul (KR); Agustina SETIAWATI, Seoul (KR); Chang Ho KIM, Seoul (KR); Soo Ryeon RYU, Seoul (KR); Kwang Hwan JUNG, Seongnam-si (KR); Bong Gun JU, Seoul (KR)

(21) Appl. No.: 17/915,669

(22) PCT Filed: Oct. 13, 2020
(86) PCT No.: PCT/KR2020/013896

§ 371 Date: Sep. 29, 2022

(30) Foreign Application Priority Data
Mar. 30, 2020 (KR) 10-2020-0038372

(51) Int. Cl. A61L 17/14; A61L 17/10; D02G 3/44; D02G 3/02; D06M 15/356; D06M 10/00; D06M 15/15

(52) U.S. Cl. CPC A61L 17/14; A61L 17/10; A61L 17/145; D02G 3/448; D02G 3/02; D06M 15/3566; D06M 10/001; D06M 15/15; A61L 2400/18; A61L 2400/10; D10B 2509/04; D06M 2101/32

(57) ABSTRACT

The present invention relates to a surgical suture having excellent biocompatibility and low friction, and to a method for manufacturing same. The suture according to the present invention has excellent biocompatibility and low friction, thereby reducing pain that may occur in a patient during suturing and minimizing the occurrence of inflammation in cells at a suture site, and also has excellent contact properties with cells, thereby capable of being used for various medical and surgical operations.

### TECHNICAL FIELD

The present invention relates to a surgical suture having excellent biocompatibility and low friction and a method for manufacturing the surgical suture and, more specifically, to a suture having high biocompatibility and low friction and thus capable of minimizing the inflammatory response and pain that may occur in the cell tissue of a patient, and a method for manufacturing the suture.

### BACKGROUND ART

A suture refers to a thread used to sew a wound caused by external injury or a part of the human body damaged by surgery or the like.

In the beginning, catgut made from extracts of organs, such as intestines and tendons of sheep, pigs, and horses, was widely used as sutures, but the use of the catgut was unsuitable due to low strength thereof and gradually decreased due to the occurrence of tissue rejection and animal-derived diseases.

Thereafter, alternative materials, such as nylon and silk, which are synthetic polymers, were developed and widely used, but such materials were not decomposed in the human body and had poor biocompatibility, causing the inconvenience of having to be additionally removed when a certain period of time elapsed after use.

Since the 1970s, research has been steadily conducted on the development and utilization of synthetic polymer sutures containing ester bonds that are decomposed by long-term exposure under water-soluble conditions, and in recent years, polydioxanone (PDO) polymers and the like have been widely used as suture materials.

Polydioxanone sutures have been mainly used for medical surgery due to their degradability. However, absorbent sutures, such as polydioxanone having ester bonds, had problems in that they had a risk of side effects resulting from foreign body rejection occurring at the initial time of use and their residues remained in the body for a long period of time even though they were degraded by the body fluid or the like in the body. Moreover, the sutures with high frictional force on the surface thereof caused inflammatory response or severe pain in the skin tissue of a patient during a suture procedure.

There is recently an increasing need for sutures that cause less pain during suturing due to low friction on the surface thereof, minimize the inflammatory response due to friction with tissue cells, and cause no side effects through high biocompatibility even when remaining in the body after a certain period of time.

### DISCLOSURE OF INVENTION

### Technical Problem

The present inventors manufactured sutures having a surface modified with ultraviolet light and/or polystyrene sulfonate (PSS) and sutures with fibronectin adsorbed onto surfaces thereof, and confirmed that the sutures according to the present invention had low friction, minimized side effects such as inflammation, and superior biocompatibility.

Accordingly, an aspect of the present invention is to provide sutures having a surface modified with ultraviolet light and/or polystyrene sulfonate and sutures with fibronectin adsorbed onto surfaces thereof.

Another aspect of the present invention is to provide methods for manufacturing sutures having a surface modified with ultraviolet light and/or polystyrene sulfonate and sutures with fibronectin adsorbed onto surfaces thereof.

### Solution to Problem

The present invention is directed to a surgical suture having excellent biocompatibility and low friction and a method for manufacturing the surgical suture, and the suture according to the present invention has effects of causing less pain and minimizing the inflammatory response that may occur in the cell tissue when used for a suture procedure.

The present inventors confirmed that the sutures according to the present invention had a high adsorption rate of fibronectin, small friction on the surface thereof due to high wettability and hydrophilicity, and excellent biocompatibility.

Hereinafter, the present invention will be described in more detail.

In accordance with an aspect of the present invention, there is provided a surgical suture in which the surface of a yarn body is modified by treatment of the yarn body with ultraviolet light or polystyrene sulfonate (PSS).

In an embodiment of the present invention, in the suture, the surface of a yarn body may be modified by treatment of the yarn body with ultraviolet light.

In the present invention, the treatment with ultraviolet light may be an ultraviolet treatment method that is commonly used in the art, and for example, may be an ultraviolet-ozone treatment method using UV-ozone plasma, but is not limited thereto.

In one embodiment of the present invention, in the suture, the surface of the yarn body may be modified by ultraviolet-ozone treatment.

In the suture according to the present invention, the surface of the suture may be modified into a surface containing —OH group (hydrophilic functional group) through ultraviolet-ozone treatment.

In an embodiment of the present invention, in the suture, the surface of the yarn body may be modified by treating the yarn body with polystyrene sulfonate.

In the present invention, polystyrene sulfonate may be a compound represented by chemical formula 1 below.

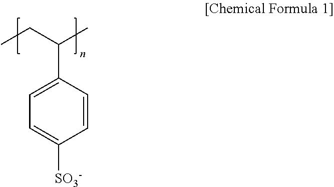

In the present invention, the suture may be treated with polystyrene sulfonate mixed with a solvent.

In the present invention, the solvent may be water, an organic solvent, or a mixture thereof, and for example, at least one selected from the group consisting of DMF, acetone, a lower alcohol having 1 to 6 carbon atoms, ethyl acetate, methylene chloride, and chloroform, but is not limited thereto.

In the present invention, the lower alcohol having 1 to 6 carbon atoms may be at least one selected from the group consisting of methanol, ethanol, propanol, butanol, normal-propanol, iso-propanol, normal-butanol, 1-pentanol, 2-butoxyethanol, and ethylene glycol, but is not limited thereto.

In the present invention, an excessively high concentration of polystyrene sulfonate to be used to treat the suture may excessively increase the amount of the coating solution sticking to the surface of the suture during coating and increase the viscosity of the coating solution, resulting in difficulty in coating, but a low concentration of polystyrene sulfonate may result in unfavorable adsorption of fibronectin. Therefore, the concentration of polystyrene sulfonate may be selected appropriately depending on the suture, and may be for example, 0.01 to 99.9%, 1 to 99%, 10 to 90%, 15 to 60%, 20 to 50%, 25 to 40%, 28 to 35%, or 30%, but is not limited thereto.

In an embodiment of the present invention, the treatment with polystyrene sulfonate may be performed by a method of dip coating, dipping, spraying, wiping, or brushing, and for example, may be performed by dip coating, but is not limited thereto.

In the present invention, the contact angle of the suture with the modified surface with respect to a water-soluble solution may be 95 degrees or lower, 90 degrees or lower, 85 degrees or lower, 80 degrees or lower, 75 degrees or lower, 70 degrees or lower, or 65 degrees or lower, and for example, 90 degrees or lower, but is not limited thereto.

In the present invention, the water-soluble solution may be at least one selected from the group consisting of water, saline solution, and serum, but is not limited thereto.

In one embodiment of the present invention, the yarn body may be at least one selected from the group consisting of a braided yarn, a monofilament, and a multifilament, and for example, may be a monofilament, but is not limited thereto.

In the present invention, the yarn body of the suture may include at least one selected from the group consisting of silk fibroin, polydioxanone, polypropylene, polyglactin, nylon, catgut, and poly glycolic acid, and for example, may include polydioxanone, but is not limited thereto.

In an embodiment of the present invention, the suture may further include a fibronectin layer on the modified surface of the suture.

As used herein, the term “fibronectin” refers to a high molecular weight (about 440 kDa) glycoprotein of an extracellular matrix binding to integrin as well as other extracellular matrix proteins, wherein the fibronectin is a protein that plays a crucial role in cell adhesion, migration, and differentiation.

In an embodiment of the present invention, the amount of fibronectin adsorbed in the fibronectin layer may be 15 to 100 ng/mm², 20 to 90 ng/mm², 25 to 80 ng/mm², 30 to 75 ng/mm², 35 to 70 ng/mm², or 40 to 60 ng/mm², and for example, may be 30 to 60 ng/mm², but is not limited thereto.

In an embodiment of the present invention, the fibronectin layer may be formed by adsorption of fibronectin onto the suture with the modified surface through dipping, spraying, wiping, brushing, or the like, and for example, dipping, but is not limited thereto.

In another aspect of the present invention, there is provided a method for manufacturing a surgical suture, the method including:

a preparation step of preparing a yarn body; and

a modification step of modifying the surface of the yarn body by treating the yarn body with ultraviolet light or polystyrene sulfonate (PSS).

In an embodiment of the present invention, the yarn body may be at least one selected from the group consisting of a braided yarn, a monofilament, and a multifilament, and for example, may be a monofilament, but is not limited thereto.

In the present invention, the yarn body in the preparation step may include at least one selected from the group consisting of silk fibroin, polydioxanone, polypropylene, polyglactin, nylon, catgut, and poly glycolic acid, and for example, may include polydioxanone, but is not limited thereto.

In the present invention, the contact angle of the suture with the modified surface with respect to a water-soluble solution may be 95 degrees or lower, 90 degrees or lower, 85 degrees or lower, 80 degrees or lower, 75 degrees or lower, 70 degrees or lower, 65 degrees or lower, and for example, 90 degrees or lower, but is not limited thereto.

In the present invention, the water-soluble solution may be at least one selected from the group consisting of water, saline solution, and serum, but is not limited thereto.

In an embodiment of the present invention, in the modification step, the surface of the yarn body may be modified by treatment of the yarn body with ultraviolet light.

In the present invention, the treatment with ultraviolet light may be an ultraviolet treatment method that is commonly used in the art, and for example, may be an ultraviolet-ozone treatment method using UV-ozone plasma, but is not limited thereto.

In an embodiment of the present invention, in the modification step, the surface of the yarn body may be modified by treatment of the yarn body with ultraviolet light.

In an embodiment of the present invention, the modification step may include a polystyrene sulfonate treatment step of treating the yarn body with polystyrene sulfonate.

In an embodiment of the present invention, the modification step may further include a curing step of curing polystyrene sulfonate, after the polystyrene sulfonate treatment step.

In the present invention, the curing step may be performed at 30 to 120° C., 40 to 110° C., 50 to 105° C., or 60 to 100° C., and for example, 65° C., but is not limited thereto.

In an embodiment of the present invention, the curing step may be performed for 4 to 12 hours, 4 to 10 hours, 4 to 8 hours, 4 to 6 hours, but is not limited thereto.

In the present invention, the suture may be treated with polystyrene sulfonate mixed with a solvent in the polystyrene sulfonate treatment step.

In the present invention, the solvent may be water, an organic solvent, or a mixture thereof, and for example, at least one selected from the group consisting of DMF, acetone, a lower alcohol having 1 to 6 carbon atoms, ethyl acetate, methylene chloride, and chloroform, but is not limited thereto.

In the present invention, the lower alcohol having 1 to 6 carbon atoms may be at least one selected from the group consisting of methanol, ethanol, propanol, butanol, normal-propanol, iso-propanol, normal-butanol, 1-pentanol, 2-butoxyethanol, and ethylene glycol, but is not limited thereto.

In an embodiment of the present invention, the concentration of polystyrene sulfonate in the polystyrene sulfonate treatment step may be 0.01 to 99.9%, 1 to 99%, 10 to 90%, 15 to 60%, 20 to 50%, 25 to 40%, 28 to 35%, or 30%, but is not limited thereto.

In an embodiment of the present invention, the treatment with polystyrene sulfonate may be performed by a method of dip coating, dipping, spraying, wiping, or brushing, and for example, may be performed by dip coating, but is not limited thereto.

In the present invention, the manufacturing method may further include an adsorption step of allowing fibronectin to be adsorbed onto the suture with the modified surface to form a fibronectin layer.

In an embodiment of the present invention, the amount of fibronectin adsorbed in the fibronectin layer may be 15 to 100 ng/mm², 20 to 90 ng/mm², 25 to 80 ng/mm², 30 to 75 ng/mm², 35 to 70 ng/mm², or 40 to 60 ng/mm², and for example, 30 to 60 ng/mm², but is not limited thereto.

In an embodiment of the present invention, the adsorption step may further include a mixing step of mixing fibronectin with at least one selected from the group consisting of phosphate buffered saline (PBS) and simulated body fluid (SBF).

In an embodiment of the present invention, fibronectin may be adsorbed onto the suture with the modified surface by a method of dipping, spraying, wiping, brushing, or the like, and for example, by dipping, but is not limited thereto.

### Advantageous Effects of Invention

The present invention relates to a surgical suture having excellent biocompatibility and low friction and a method for manufacturing the surgical suture, and the suture according to the present invention has excellent biocompatibility and low friction and thus reduces the pain that may occur in a patient during suturing, minimizes the inflammation occurring in cells of a sutured site, and has excellent contact properties to cells, and thus can be utilized in various kinds of internal and external surgery.

### BRIEF DESCRIPTION OF DRAWINGS

FIG. 1 shows images taken with a scanning electron microscope (SEM), illustrating surfaces of a polydioxanone suture (PDO suture), a fibronectin-adsorbed UV-treated suture (PDO-UV/FN suture), and a fibronectin-adsorbed PSS-treated suture (PDO-PSS/FN suture) according to an embodiment of the present invention.

FIG. 2 shows graphs illustrating the results of Fourier transform infrared (FT-IR) spectrometry of a PDO-UV/FN suture by an FT-IR spectrometer according to an embodiment of the present invention.

FIG. 3 shows graphs illustrating the results of FT-IR spectrometry of a PDO-PSS/FN suture obtained by an FT-IR spectrometer according to an embodiment of the present invention.

FIG. 4 shows a graph illustrating the amount of fibronectin adsorbed onto the surface of PDO suture, PDO-UV suture, and PDO-PSS suture according to an embodiment of the present invention.

FIG. 5 shows fluorescent microscopic 3D images of fibroblast cells cultured on fibronectin-adsorbed PDO suture (PDO-FN suture), PDO-UV/FN suture, and PDO-PSS/FN suture according to an embodiment of the present invention.

FIG. 6 shows a graph illustrating the number of fibroblast cells cultured on PDO-FN suture, PDO-UV/FN suture, and PDO-PSS/FN suture according to an embodiment of the present invention.

FIG. 7 shows a graph illustrating the extent of suturing of PDO-FN suture, PDO-UV/FN suture, and PDO-PSS/FN suture in fibroblast cells opening around the sutures according to an embodiment of the present invention.

FIG. 8 shows images illustrating the extent of suturing of PDO-FN suture, PDO-UV/FN suture, and PDO-PSS/FN suture in fibroblast cells opening around the sutures according to an embodiment of the present invention.

FIG. 9 shows graphs illustrating the results of measuring the contact angle, with respect to water, of PDO suture, PDO-UV suture, PDO-UV/FN suture, PDO-PSS suture, and PDO-PSS/FN suture according to an embodiment of the present invention.

FIG. 10 shows graphs illustrating the results of measuring the contact angle, with respect to saline solution, of PDO suture, PDO-UV suture, PDO-UV/FN suture, PDO-PSS suture, and PDO-PSS/FN suture according to an embodiment of the present invention.

FIG. 11 shows graphs illustrating the results of measuring the contact angle, with respect to serum, of PDO suture, PDO-UV suture, PDO-UV/FN suture, PDO-PSS suture, and PDO-PSS/FN suture according to an embodiment of the present invention.

### BEST MODE FOR CARRYING OUT THE INVENTION

A surgical suture in which the surface of a yarn body is modified by treatment of the yarn body with ultraviolet light or polystyrene sulfonate (PSS)

### MODE FOR CARRYING OUT THE INVENTION

Hereinafter, the present invention will be described in more detail by the following exemplary embodiments. However, these exemplary embodiments are used only for illustration, and the scope of the present invention is not limited by these exemplary embodiments.

### Example 1: Manufacturing of sutures

1-1. Manufacturing of UV-Treated Suture

To manufacture a UV (UV-ozone)-treated suture, a Monosorb® polydioxanone suture (PDO suture) product purchased from Samyang Biopharm (Korea) was used as a raw yarn for a suture.

The polydioxanone suture was irradiated with ozone plasma for 4 minutes by UV OZONE CLEANER (AhTech LTS, Korea), which is a UV ozone plasma (UVO plasma) generator equipment to introduce —OH groups to the surface of the suture, thereby manufacturing a UV-treated suture (PDO-UV suture) with a hydrophilically modified surface.

1-2. Manufacturing of Polystyrene Sulfonate-Treated Suture

A Monosorb® polydioxanone suture purchased from Samyang Biopharm (Korea) was coated with polystyrene sulfonate (PSS) by dip coating. Thereafter, to cure PSS, the surface of the suture was cured at a temperature of 65° C. for at least 4 hours in an oven, thereby manufacturing a polystyrene sulfonate surface-treated suture (PDO-PSS suture).

### Example 2: Manufacturing of Fibronectin-Adsorbed Suture and Determination of Degree of Adsorption

2-1. Manufacturing of Fibronectin-Adsorbed Suture

The product Fibronectin Human, Plasma from Thermo Fisher Scientific was used as a fibronectin (FN) to be adsorbed on the surfaces of the PDO-UV suture and PDO-PSS suture manufactured in Examples 1-1 and 1-2.

A fibronectin solution was prepared by diluting fibronectin in a PBS solution to a concentration of 50 μg/ml, and the PDO-UV suture and the PDO-PSS suture were dipped in the fibronectin solution and then incubated for 72 hours with gentle shaking in an incubator at 37° C., thereby allowing fibronectin to be well adsorbed onto the surfaces of the surface-treated sutures. After the incubation, the sutures were washed several times with a PBS solution. The suture surface analysis and FT-IR spectrometry were performed after the surfaces of the sutures were dried in air.

Besides the PDO suture, silk fibroin suture (Black Silk®, Mersilk®), polydioxanone suture (PDS®II), polypropylene suture (Prolene®), polyglactin suture (Vicryl®), nylon suture (Blue Nylon®), enteric suture (Chromic®), and polyglycolic acid suture (Surgifit®) were also subjected to fibronectin adsorption after UV treatment or PSS surface treatment in the same manner as described above.

2-2. Suture Surface Analysis

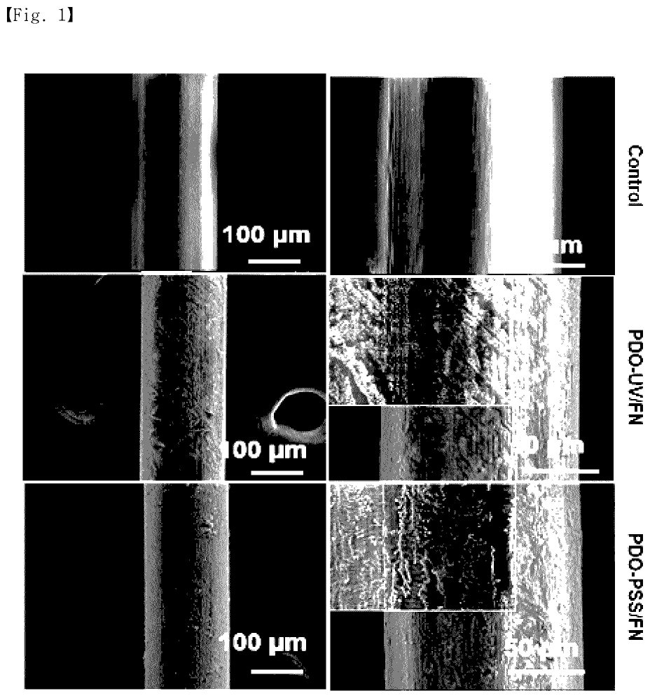

After the fibronectin adsorption, the PDO suture, fibronectin-adsorbed UV-treated suture (PDO-UV/FN suture), and fibronectin-adsorbed PSS-treated suture (PDO-PSS/FN suture) were subjected to surface observation through a scanning electron microscope (SEM), and the results are shown in FIG. 1.

As shown in FIG. 1, the PDO-UV/FN suture and the PDO-PSS/FN suture showed irregularly shaped materials attached to the surfaces of microfibrils, which was not observed in the PDO suture as a control. Therefore, it was predicted that fibronectin was attached onto the PDO-UV suture or PDO-PSS suture.

2-3. FT-IR Absorption Spectrometry

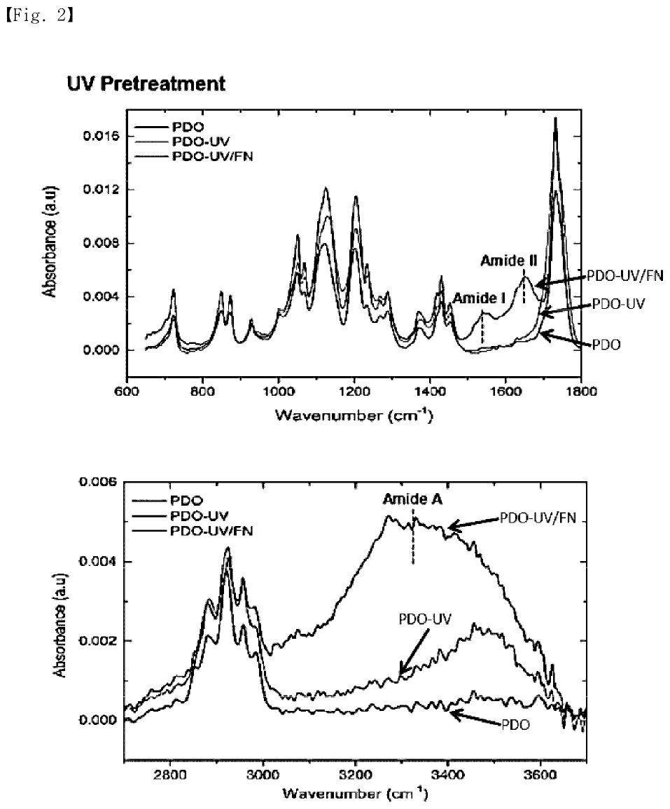

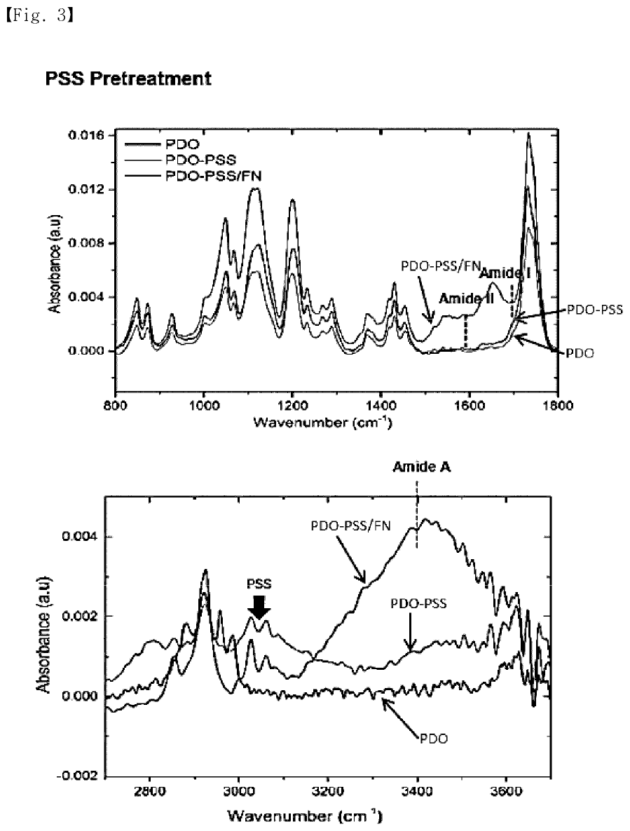

After the fibronectin adsorption, each of the PDO-UV/FN suture and the PDO-PSS/FN suture was subjected to Fourier transform (FT-IR) spectrometry through an FT-IR spectrometer. The infrared absorption spectrometry was performed with an ATR accessory (MIRacle™ Single Reflection ATR, PIKE Technologies, USA) through a total reflection method of the infrared spectrometer Cary 640 (Agilent Technologies, USA). The suture samples were placed and fixed onto the ZnSe crystal of the ATR accessory and then scanned 64 times with a resolution of 4 cm⁻¹ in a wavenumber range of 600-4000 cm-1, and average values thereof were used to obtain spectra. The results are shown in FIGS. 2 and 3.

As a result of FT-IR analysis, as shown in the graphs of FIGS. 2 and 3, the PDO-UV/FN suture and the PDO-PSS/FN suture showed absorption peaks near 1600 cm-1 due to Amide I and Amide II and an absorption peak near 3300 cm-1 due to Amide A. However, the PDO suture and the PDO-UV or PDO-PSS suture without adsorbed fibronectin did not show absorption peaks at corresponding wavenumbers.

The test results confirmed that fibronectin was well adsorbed onto the surfaces of both the PDO-UV/FN suture and the PDO-PSS/FN suture.

The silk fibroin suture (Black Silk®, Mersilk®), polydioxanone suture (PDS®II), polypropylene suture (Prolene®), polyglactin 910 suture (Vicryl®), nylon suture (Blue Nylon®), enteric suture (Chromic®), and polyglycolic acid suture (Surgifit®), onto which fibronectin was adsorbed, were subjected to FT-IR spectrometry (not shown), and it was confirmed that like in the results of the PDO-UV suture and the PDO-PSS suture, fibronectin was well adsorbed onto the surfaces of those sutures.

2-4. Adsorption Amount of Fibronectin

To determine the accurate adsorption amount of fibronectin, 2-cm long samples of the PDO suture, PDO-UV suture, and PDO-PSS suture were prepared, and fibronectin was adsorbed onto the surface of each suture by the same method as in Example 2-1.

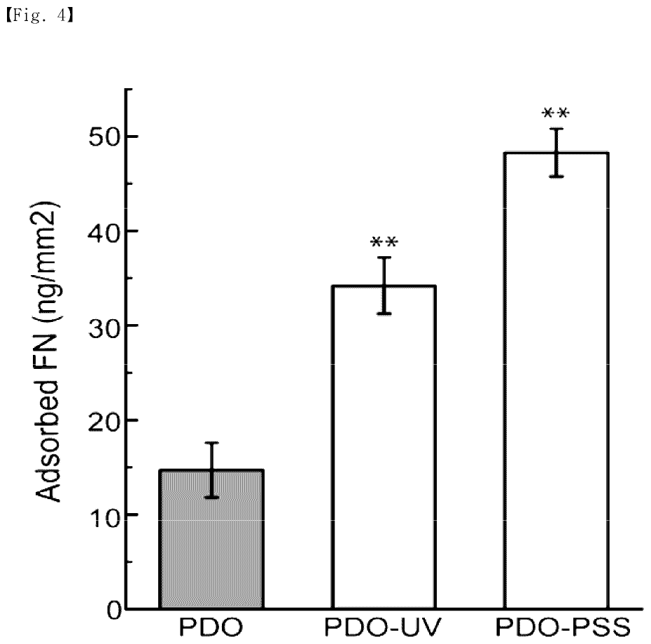

The adsorption amount of fibronectin was quantified using the Pierce modified Lowry method, which is a protein quantification method, and in the fibronectin absorption process, fibronectin solutions before and after incubation were collected in 96-well plates. After 200 μl of a Modified Lowry reagent was added to each of the collected fibronectin solutions, followed by gently shaking at room temperature for 10 minutes, and additionally, the Folin-Ciocalteu reagent was mixed therewith, followed by shaking for 30 seconds. Thereafter, the 96-well plates were blocked from light by using an aluminum foil and incubated at room temperature for 30 minutes. Then, the absorbance of each well was measured at a wavelength of 750 nm by using a multiple reader (EnSpire, PerkinElmer), and the concentration of fibronectin protein was determined through a quantitative absorbance standard curve. The concentration of the adsorbed fibronectin was determined by comparing the concentrations of the solution before and after fibronectin absorption, and the adsorption amount was expressed in ng/mm² by dividing the concentration of the adsorbed fibronectin by a surface area of the suture sample. The results are shown in FIG. 4 and Table 1 below.

TABLE 1

| | PDO | PDO-UV | PDO-PSS |
|---|---|---|---|
| Adsorption | 14.70 ± 1.12 | 34.22 ± 1.15 | 48.28 ± 0.98 |
| amount of | | | |
| fibronectin | | | |
| (ng/mm²) | | | |

As shown in FIG. 4 and Table 1, the adsorption amount of fibronectin was significantly increased in the PDO-UV suture and the PDO-PSS suture compared with the control PDO suture.

These results confirmed that fibronectin was adsorbed onto the surfaces of the PDO-UV suture and the PDO-PSS suture, with excellent adsorptive power. Especially, the adsorptive power of fibronectin onto the PDO-PSS suture was the highest.

It was therefore assumed that the surface treatment of a suture through UV or polystyrene sulfonate increased the adsorptive power of fibronectin onto the surface of the suture.

### Example 3: Biocompatibility

3-1. Cell Density

A surface of a culture dish was coated with 5% Pluronic™ F127 solution, washed with DI water, and disinfected with ultraviolet light. Thereafter, a culture medium and a PDO suture, a PDO-UV suture, or a PDO-PSS suture were placed in the disinfected culture dish and GFP fluorescence-expressing fibroblast cells were seeded. A sample group in which fibroblast cells were cultured for 24 hours and a sample group in which fibroblast cells were cultured for 72 hours were prepared.

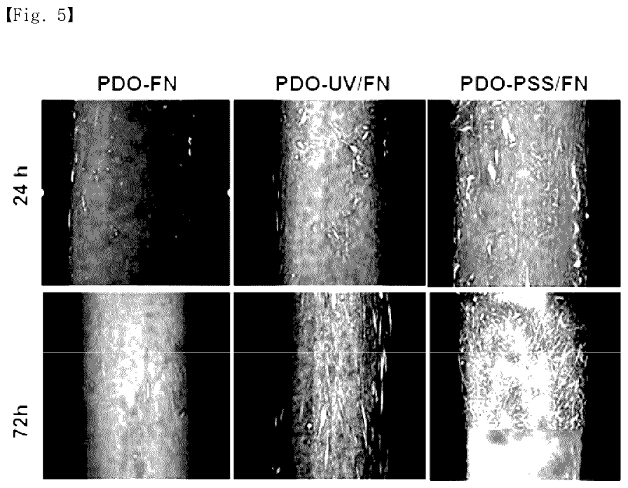

After incubation, the suture was isolated from the culture dish and transferred to a culture dish containing a new culture solution. Thereafter, fibroblast cells that were grown and expressed in green on the suture were measured as Z-stack images by a confocal microscope equipment (TCS SP8, Leica), thereby obtaining 3D images of the suture. The obtained confocal fluorescent microscopic 3D images are shown in FIG. 5.

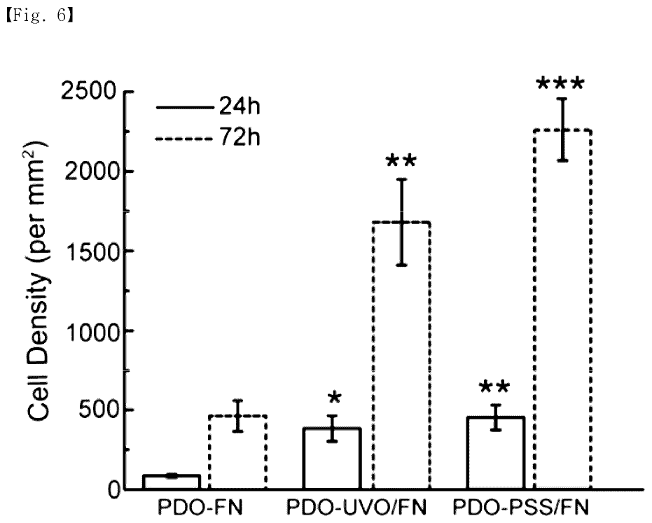

From the confocal fluorescence microscopic 3D image results, the number of the fibroblast cells in the suture was measured using the 3D object counter plug-in for ImageJ (Fiji, Japan) software. Therefore, the cell densities on the surfaces of the fibronectin-adsorbed polydioxanone yarn (PDO-FN suture), PDO-UV/FN suture, and PDO-PSS/FN suture were determined, and the results are shown in FIG. 6 and Table 2.

TABLE 2

| Cell density | | | |
|---|---|---|---|
| (per mm²) | PDO-FN | PDO-UV/FN | PDO-PSS/FN |
| 24 hours | 86.89 ± 8.25 | 384.14 ± 80.61 | 447.16 ± 77.81 |
| culture | | | |
| 72 hours | 461.98 ± 96.77 | 1680.69 ± 269.72 | 2258.71 ± 193.62 |
| culture | | | |

As can be confirmed in FIGS. 5 and 6 and Table 2, the cell densities measured on the PDO-UV/FN suture and PDO-PSS/FN suture were significantly higher than those of the control PDO-FN suture.

3-2. Cell Migration/Cell Healing

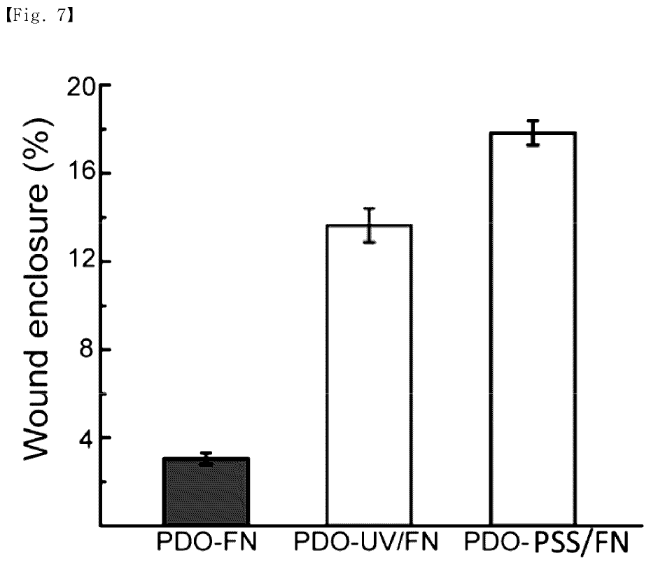

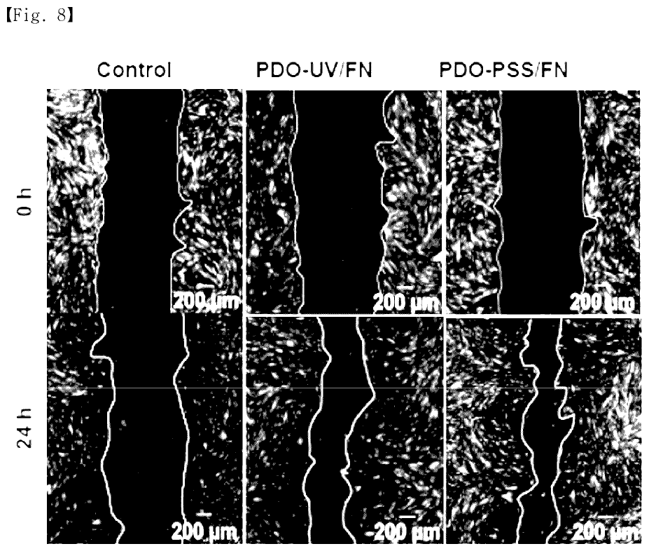

Fibroblast cells (confluent fibroblasts) were plated by culture and growing on a plate, and then a groove with a diameter of 1 mm was formed in the center of the plated fibroblast cells by using a cell scraper. Thereafter, each of the PDO-FN suture, the PDO-UV/FN suture, and the PDO-PSS/FN suture was placed in the groove formed in the center of the fibroblast cells, and the fibroblast cells were again cultured with low-serum media. The degree of enclosure of the opened fibroblast cells was measured and compared at 0 hour and 24 hours after the culture, and the results are shown in FIGS. 7 to 8 and Table 4.

TABLE 4

| | | | PDO- |
|---|---|---|---|
| | PDO-FN | PDO-UV/FN | PSS/FN |
| Cell | 3.05 ± 0.25 | 13.64 ± 0.77 | 17.83 ± 0.55 |
| enclosure | | | |
| (%) | | | |

As can be confirmed from FIG. 7, the degrees of cell migration/cell healing in the PDO-UV/FN suture and PDO-PSS/FN suture were 4-fold higher than that in the PDO-FN suture.

These results confirmed that the PDO-UV/FN suture and PDO-PSS/FN suture were excellent compared with the PDO-FN suture in terms of both cell culture density and cell migration (healing) on the surface of a suture.

Therefore, both the PDO-UV/FN suture and the PDO-PSS/FN suture were confirmed to have excellent biocompatibility, and an application of the sutures according to the present invention is expected to attain a stable combination between a wound site and adjacent cell tissue and the sutures and minimize a rejection reaction in the surrounding tissues.

### Example 4: Physicochemical Properties

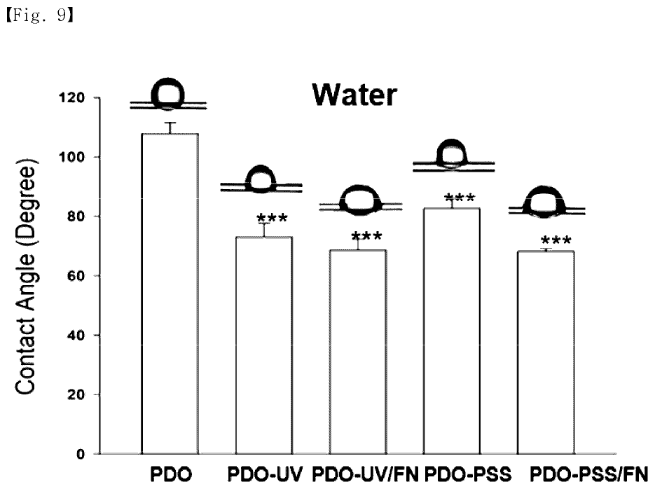

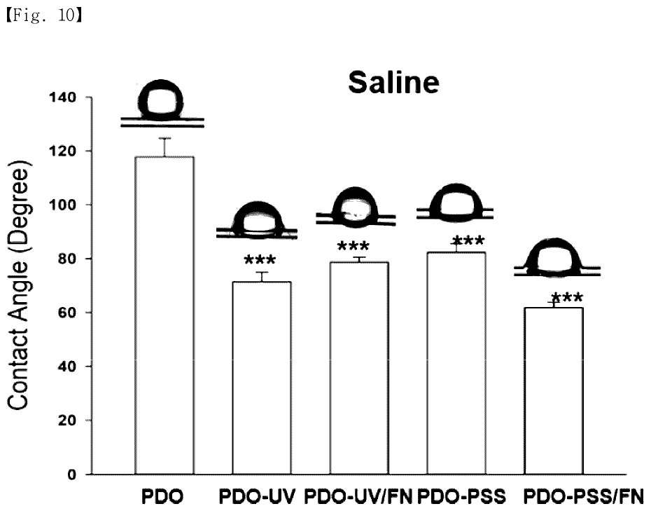

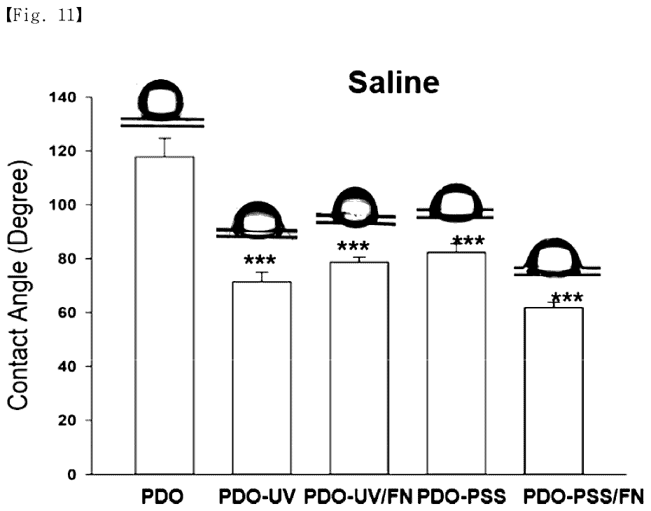

To determine the hydrophilicity and wettability of the sutures, the PDO suture as a control, PDO-UV suture, PDO-UV/FN suture, PDO-PSS suture, and PDO-PSS/FN suture were measured for contact angles (θ) with respect to water, saline solution, and serum by using the contact angle meter Phoenix 500 (SEO, Korea). Drops of water, saline solution, and serum were formed on each suture, and then imaged by a CCD camera to measure contact angles through the obtained images, and the results are shown in FIGS. 9 to 11 and Table 5.

TABLE 5

| | | PDO- | PDO- | PDO- | PDO- |
|---|---|---|---|---|---|
| Contact angle | PDO | UV | UV/FN | PSS | PSS/FN |
| Water (°) | 107.8 | 73.1 | 68.6 | 82.7 | 68.2 |
| Saline solution | 117.8 | 71.4 | 78.7 | 82.3 | 61.9 |
| (°) | | | | | |
| Serum (°) | 97.1 | 69.8 | 67.9 | 65.2 | 50.7 |

In general, a material to be measured is assumed to have hydrophobicity when having a contact angle of 90 degrees or higher, and hydrophilicity when having a contact angle of 90 degrees or lower. As a result of the test, the PDO suture had contact angles of 90 degrees or higher with respect to all of water, saline solution, and serum, indicating that the surface of the suture is hydrophobic. However, the PDO-UV, PDO-UV/FN, PDO-PSS, and PDO-PSS/FN sutures had contact angles of 90 degrees or lower with respect to all of water, saline solution, and serum. It could be therefore confirmed that the surface of a suture was changed from hydrophobicity to hydrophilicity and wettability by surface treatment.

These results confirmed that all the PDO-UV, PDO-UV/FN, PDO-PSS, and PDO-PSS/FN sutures had excellent hydrophilicity and wettability. These results also confirmed that the sutures have little friction upon contact with cellular tissues of the human body.

Therefore, the PDO-UV suture, PDO-UV/FN suture, PDO-PSS suture, PDO-PSS/FN suture are expected to, due to their low friction, reduce the patient's pain during wound suturing and minimize the inflammatory response of cell tissue that may occur during suturing.

### INDUSTRIAL APPLICABILITY

The present invention relates to a surgical suture having excellent biocompatibility and low friction and a method for manufacturing the surgical suture, more specifically, to a suture having high biocompatibility and low friction and thus capable of minimizing the inflammatory response and pain that may occur in the cell tissue of a patient, and a method for manufacturing the suture.

1. A surgical suture in which the surface of a yarn body is modified by treatment of the yarn body with ultraviolet light or polystyrene sulfonate (PSS).

2. The surgical suture of claim 1, wherein the surface of the yarn body is modified by ultraviolet-ozone treatment.

3. The surgical suture of claim 1, wherein the yarn body includes polydioxanone (PDO).

4. The surgical suture of claim 1, further comprising a fibronectin layer on the modified surface of the suture.

5. The surgical suture of claim 4, wherein the amount of fibronectin adsorbed onto the fibronectin layer is 30 to 75 ng/mm².

6. The surgical suture of claim 1, wherein the suture with the modified surface has a contact angle of 90 degrees or lower with respect to a water-soluble solution.

7. A method for manufacturing a surgical suture, the method comprising:
a preparation step of preparing a yarn body; and
a modification step of modifying the surface of the yarn body by treating the yarn body with ultraviolet light or polystyrene sulfonate (PSS).

8. The surgical suture of claim 7, wherein in the modification step, the surface of the yarn body is modified by ultraviolet-ozone treatment.

9. The method of claim 7, wherein the yarn body includes polydioxanone (PDO).

10. The method of claim 7, further comprising an adsorption step of allowing fibronectin to be adsorbed onto the suture with the modified surface to form a fibronectin layer.

11. The method of claim 10, wherein in the adsorption step, the amount of fibronectin adsorbed in the fibronectin layer is 30 to 75 ng/mm².
